(19) United States
(12) Patent Application Publication
(10) Pub. No.: US 2020/0032288 A1
(43) Pub. Date: Jan. 30, 2020

(54) METHOD OF ENHANCING STRESS TOLERANCE OF MONOCOTYLEDONOUS PLANTS

(71) Applicant: STELLENBOSCH UNIVERSITY, Stellenbosch (ZA)

(72) Inventors: Anna-Maria BOTHA-OBERHOLSTER, Stellenbosch (ZA); Christell VAN DER VYVER, Somerset West (ZA); Marlon Luke LE ROUX, Kuilsrivier (ZA)

(21) Appl. No.: 15/740,049

(22) PCT Filed: Jun. 30, 2016
(86) PCT No.: PCT/IB2016/053936

§ 371 Date: Dec. 27, 2017

(30) Foreign Application Priority Data
Jun. 30, 2015 (ZA) 2015/04699

(51) Int. Cl. C12N 15/82

(52) U.S. Cl. CPC C12N 15/8273

(57) ABSTRACT

A method is provided for producing a transformed plant of the family Poaceae. One or more genes selected from the group consisting of OTS1, OTS2 and ICE1 are introduced into the genome of the plant, resulting in transformed plants having enhanced tolerance to an abiotic stress compared to untransformed plants. The gene can be under the control of a drought inducible promoter, such as the Rab17 promoter. Examples of the abiotic stress are drought, heat, cold and salinity. The plant can be a cereal or grass, such as crops or forage grasses including wheat, barley, sorghum, maize, sugarcane, oats, rye, triticale and commercial fodder grass species. The invention also extends to a vector for transforming the plants and to transformed plants and plant parts.

### CROSS-REFERENCE TO RELATED APPLICATIONS

This application claims priority from South African provisional patent application number 2015/04699 filed on 30 Jun. 2015, which is incorporated by reference herein.

### FIELD OF THE INVENTION

The invention provides methods for enhancing the tolerance of monocotyledonous crops to abiotic stresses such as salt (high salinity), drought, heat and cold.

### BACKGROUND OF THE INVENTION

Abiotic stresses, defined as the negative impact of non-living factors on the living organisms in a specific environment, are the primary causes of crop loss for crop species such as wheat and other crops in the grass family Poaceae (e.g. maize, rice, sorghum and sugarcane). Abiotic stresses include high and low temperatures, salinity, drought, flooding, heavy metal stress and many other environmental factors.

Wheat (Triticum aestivum L.) is one of the main cereal crops grown worldwide providing essential proteins and carbohydrates to the human diet (Feuillet et al., 2007). Global wheat production in the 2012/13 growth season was 660 million tons with major producers being China, India and the USA (FAOStat, 2013). In sub-Saharan Africa, South Africa is the second largest wheat producing country after Ethiopia with production standing at 1.8 million tons in the 2014 season according to the Mundi index (www.indexmundi.com/agriculture/country). However, the wheat industry has been declining in South Africa over the past two decades due to a number of environmental factors such as inconsistent precipitation levels as well as biotic factors such as bollworms, aphids and mite infestation (van der Vyver, 2013).

Traditional approaches to breeding crop plants with improved abiotic stress tolerances have so far met with limited success. This is due to a number of contributing factors, including: (1) the focus has been on yield rather than on specific traits; (2) the difficulties in breeding for tolerance traits; and (3) desired traits can only be introduced from closely related species.

Most cereals and grasses are moderately sensitive to a wide range of abiotic stresses, and variability in the gene pool generally appears to be relatively small and may provide few opportunities for major step changes in tolerance. Of potentially larger impact on abiotic stress tolerance is the use of genetic manipulation technologies to generate such step changes.

Arabidopsis (Arabidopsis thaliana) is often used as a model plant for plant transformation. However, knowledge gained from working on Arabidopsis is not particularly suitable for transfer to the grasses (including both cereals and forage species). In addition to the obvious fundamental differences in development and anatomy between monocotyledons and dicotyledons, many of the mechanisms of tolerance to abiotic stresses can have fundamentally different characteristics between these two major plant groups. For example, when grown in saline soils, many dicotyledonous halophytes accumulate much higher concentrations of Na+ in their shoots than monocotyledonous halophytes, a feature that may be related to the observation that succulence is observed more commonly in dicotyledons than monocotyledons, particularly the grasses.

The possibilities for increasing tolerance to abiotic stresses are enormous, although it is notable that the actual production of transgenic plants with demonstrably improved abiotic stress tolerance has been slow. There have been many published reviews outlining the possibilities for inducing stress tolerance, but, to date, only a handful of papers have been published presenting results from transgenic plants. Unfortunately, some of the transgenic alterations appear to come with a growth and/or yield penalty in conditions of reduced stress (Tester and Bacic, 2005).

Thus, there is still a need to provide cereal and grass crops with increased abiotic stress tolerance.

### SUMMARY OF THE INVENTION

According to a first embodiment of the invention, there is provided a method for producing a transformed plant of the family Poaceae, the method comprising the step of introducing one or more nucleic acids encoding genes selected from the group consisting of OTS1, OTS2 and ICE1 into the genome of the plant, wherein the transformed plant has enhanced tolerance to an abiotic stress compared to an untransformed plant.

The plant may be a cereal or grass, such as crops or forage grasses including wheat, barley, sorghum, maize, sugarcane, oats, rye, triticale and commercial fodder grass species. More preferably, the plant is wheat or sugarcane.

The abiotic stress may be drought, heat, cold or salinity.

The gene may be under the control of a drought inducible promoter, such as the Rab17 promoter.

The nucleic acid may have a nucleotide sequence which is at least 80% identical to SEQ ID NO. 3, at least 80% identical to SEQ ID NO. 9 or at least 80% identical to SEQ ID NO. 11.

According to a second embodiment of the invention, there is provided vector for transforming a plant as described above, the vector comprising one or more genes selected from the group consisting of OTS1, OTS2 and ICE1 under the control of a Rab17 promoter.

According to a third embodiment of the invention, there is provided a transformed plant or plant part produced according to the method described above. The transformed part plant may be a seed, plantlet, leaf or the like.

### BRIEF DESCRIPTION OF THE FIGURES

FIG. 1: Donor plant cultivar Gamtoos R (Dn7+) and Gamtoos S (Dn7−) in a glasshouse at 24° C. with natural light. A) Plant at 2 months, B) Plant begins anthesis, C) Caryopses collected 12-16 days post-anthesis.

FIG. 2: Vector map of the pGFP 510b (6167 bp) vector containing the green fluorescent protein gene (gfp) under the control of a maize ubiquitin promoter and cauliflower mosaic virus terminator (CaMV-t), used to assess bombardment parameters.

FIG. 3: Sequence of ICE with Rab17 promotor (SEQ ID NO. 1), where underlined region=Rab17-promoter region (647 bp; SEQ ID NO. 2) and non-underlined region=ICE gene CDS (1485 bp, SEQ ID NO. 3).

FIG. 4: Explants from experiments 1-6 expressing gfp (see Table 1 for quantitative data which correlate with this figure).

FIG. 5: Amplification of OTS1 (1.7 kb), OTS2 (1.7 kb) and ICE (1.5 kb) using Phusion High-Fidelity DNA Polymerase. Ladder used Thermo Scientific O'GeneRuler 1 kb DNA Ladder.

FIG. 6: Vector maps of plant expression vector, pUbi510 and selection vector, pEmuKN.

FIG. 7: Nest PCR to amplify Rab17 (650 bp) drought promoter from maize callus. The promoter was confirmed by sequencing.

FIG. 8: Vector map of the initial pAHC20 vector prior to the removal of its endogenous promoter (UBI) and reporter gene (Bar).

FIG. 9: Vector map of pAHC20 contain Rab17 promoter in-frame with ICE1 gene.

FIG. 10: Particle bombardment of immature embryos with WP (5 μg/μl) with an osmoticum treatment for 16 hours, and helium pressure at 80 kPa. A) Immature embryos (1 mm), B) 2-6 day old callus (3 mm), C) Individual cells expressing GFP, D) Immature embryos expressing GFP visualization with overlaying filters.

FIG. 11: Transgene confirmation of OTS1. A) Confirming stable expression of OTS1 in wheat, where (P+): Positive control (pUbi510:OTS1 plasmid); N(−): negative control; (NT): non-transformed plant; (P1-P3): transformed plant. Only P3 tested positive for transgene integration, B) The plant which tested positive (P3).

FIG. 12: Full length cDNA sequence of OTS1 (AT1G60220) (SEQ ID NO. 4). Bold and red indicates the coding sequence (SEQ ID NO. 9), underlined letters are areas on which primers were designed. Italicised letters are start and stop codons.

FIG. 13: Full length cDNA sequence of ICE1 (AT3G26744.4) (SEQ ID NO. 5). Bold and red indicate the coding sequence (SEQ ID NO. 10), underlined letters are areas on which primers were designed. Italicised letters are start and stop codons.

FIG. 14: Full length cDNA sequence of OTS2 (AT1G10570) (SEQ ID NO. 6). Bold and red indicate the coding sequence (SEQ ID NO. 11), underlined letters are areas on which primers were designed. Italicised letters are start and stop codons.

FIG. 15: Sugarcane tissue ready for hardening off.

FIG. 16: (A) Four OTS1 transgenics (1-4); (B) two OTS2 transgenics and; (C) one ICE1 transgenic. P (+) refers to plasmid DNA used for bombardment; W refers to non-transformed wheat, N negative water control.

FIG. 17: Phenotypes of spikelet and wheat plants. (A) Spikelet of (pUBI-ICE1); (B) Control spikelet; and (C) transgenic plants. Note a single plant was chosen as a representative for this photo. However there is only one transgenic plant for pUBI-ICE1

FIG. 18: (A-C) OTS1 transgenic plants; (D-E) OTS2 transgenic plants

FIG. 19: Levels of peroxidase activity measured in the transgenic and control (untransformed) plants, where plants 1 to 6 represent transgenic lines and plant 7 the untransformed control wheat plant.

FIG. 20: Levels of Glutathion-S-transferase activit; measured in the transgenic and control (untransformed) plants, where plants 1 to 6 represent transgenic lines and plant 7 the untransformed control wheat plant.

FIG. 21: Levels of β-1,3-Glucanase activity measured in the transgenic and control (untransformed) plants, where plants 1 to 6 represent transgenic lines and plant 7 the untransformed control wheat plant.

### DETAILED DESCRIPTION OF THE INVENTION

The invention provides a method for producing a transformed plant of the family Poaceae. One or more nucleic acids encoding genes selected from the group consisting of OTS1, OTS2 and ICE1 transformed into the genome of the plant, and expression of these genes in the mature plant confers upon the plant enhanced tolerance to an abiotic stress such as heat, cold, drought and/or salinity. These genes were originally isolated from Arabidopsis thaliana.

The plant is a grass or cereal, such as crops or forage grasses including wheat, barley, sorghum, maize, sugarcane, oats, rye, triticale, commercial fodder grass species and the like. In one embodiment, the plant is wheat, a hexaploid monocotyledonous food crop. In another embodiment, the plant is sugarcane.

The gene is typically under the control of an inducible promoter, and in particular a drought inducible promoter, such as the Rab17 promoter.

In an embodiment of the invention, the plant is transformed with only one of the genes, such as any one of OTS1, OTS2 or ICE1. The nucleic acid encoding the OTS1 gene can have a nucleotide sequence which is at least 80% identical to SEQ ID NO. 9, at least 80% identical to SEQ ID NO. 9, at least 90% identical to SEQ ID NO. 9, at least 95% identical to SEQ ID NO. 9 or may even be identical to SEQ ID NO. 9. The nucleic acid encoding the OTS2 gene can have a nucleotide sequence which is at least 80% identical to SEQ ID NO. 11, at least 80% identical to SEQ ID NO. 11, at least 90% identical to SEQ ID NO. 11, at least 95% identical to SEQ ID NO. 11 or may even be identical to SEQ ID NO. 11. The nucleic acid encoding the ICE1 gene can have a nucleotide sequence which is at least 80% identical to SEQ ID NO. 3, at least 80% identical to SEQ ID NO. 3, at least 90% identical to SEQ ID NO. 3, at least 95% identical to SEQ ID NO. 3 or may even be identical to SEQ ID NO. 3.

or at least 80% identical to SEQ ID NO. 11.

at least 80% identical to SEQ ID NO. 9

Alternatively, the plant can be transformed with two of the genes, such as OTS1 and ICE1, OTS2 and ICE1 or OTS1 and OTS2. More preferably, the plant is transformed with OTS1 and ICE1 or OTS2 and ICE1.

The transformation is typically performed by way of particle bombardment. Preferred conditions under which the plants are transformed include:
A circular plasmid is used;
The whole plasmid is used rather than a minimal cassette;
Transgenic DNA is delivered to the plant under a pressure of less than 90 kPa and more preferably at about 80 kPa; and
After bombardment, the plants are subjected to osmoticum treatment on non-metabolisable osmotica including sorbitol and mannitol for a maximum period of 16 hours.

The invention will now be described in more detail by way of the following non-limiting examples.

### EXAMPLE 1

### Transformation of Wheat to Enhance Stress Tolerance

Gene Identification, Vector Construction and Delivery

The most commonly used methods for wheat transformation include polyethylene glycerol (PEG)-mediated pollen tube pathway Agrobacterium tumefaciens-mediated or biolistics techniques (Yi et al., 2015). However, the latter remains the most frequently used method globally. Biolistics, also referred to as particle bombardment (PB), has achieved stable expression of various transgenes in wheat through the application of this technique (Southgate et al., 1995; Rasco-Gaunt et al., 1999; Folling, 2001; Liu et al., 2011; Jackson et al., 2013). It involves the use of a helium driven system which propels DNA-coated tungsten or gold particles into target tissue, leading to the establishment of mature transgenic plants. PB is relatively genotype-dependant, and for this reason various parameters need to be optimized (Lacock, 1990). These parameters include explants pre- and post-treatment, DNA format and quantity, propellant force, target distance and helium pressure leading to effective DNA delivery. However, this technique is not without its pitfalls (Lacock, 1990; Southgate et al., 1995), one of which is tissue damage and also the integration of complex trans-gene patterns, which ultimately lead to difficulties in subsequent analysis. Thus, prior to engineering wheat, regardless of the method of choice to introduce a foreign gene, it is of great importance to fully establish the platform, which entails extensive optimization (Ziemienowicz, 2013). This section therefore addresses the optimization in the context of particle bombardment for transient expression. These results are then mimicked to obtain stable expression in wheat.

Materials and Methods

Donor Plant and Explant Isolation and Initiation

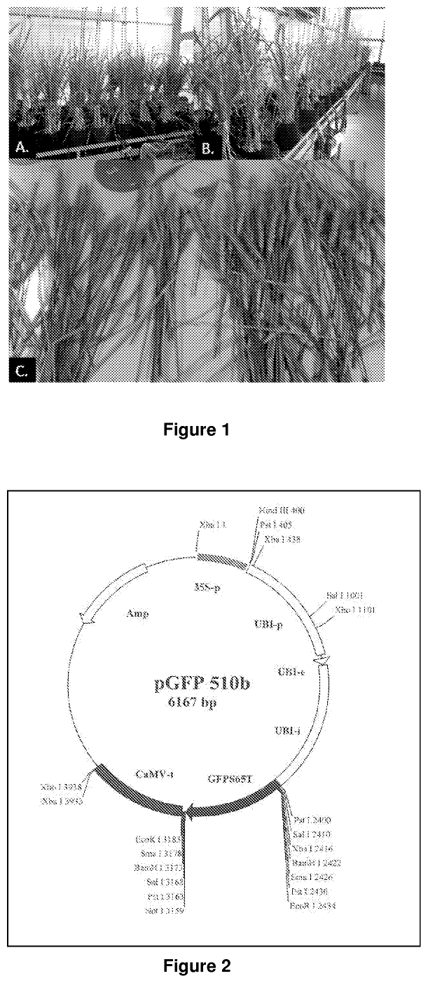

Due to superior in vitro regeneration capabilities, the cultivars Gamtoos R (Dn7+) and Gamtoos S (Dn7−) were chosen for the establishment of the engineering platform. The cultivars were grown in soil mixed with vermiculite and sand (ratio of 1:1:1) until maturity (3 months) in a glasshouse at 24° C. with natural light (FIG. 1a-b). Caryopses were collected 12-16 days post-anthesis (FIG. 1c) and surface sterilized for 15 minutes in 20% (v/v) commercial bleach (sodium hypochlorite) and 2 minutes in 70% (v/v) ethanol, containing 0.01% (v/v) Tween-20, and then rinsed with sterilised distilled water. Immature embryos were extracted under sterile conditions with the aid of a binocular microscope. The scutella was removed from the immature embryo axis, and placed on callus-embryogenic tissue induction media (INDUC A). Explants were maintained for 4-5 days at 25° C. in the dark on INDUC A media. A total of ±433 immature embryos were used and divided onto ten plates with a minimum of ±30 immature embryos per plate.

Tissue Pre-Treatment

Prior to bombardment, explants underwent turgidity treatments (osmoticum) by exposure to sorbitol (0.2 M) and mannitol (0.2 M), within basal MS medium (pH 5.6) (Southgate et al., 1995; Rivera et al., 2012). Subsequently, the most efficient turgidity treatment was optimised with respect to the duration of exposure (3, 4 or 16 hours, respectively).

Plasmid Construct and Purification

Transient Expression and PB Optimization

A pGFP 510b (6167 bp) vector containing the green fluorescent protein gene (gfp) under the control of a maize ubiquitin promoter and cauliflower mosaic virus terminator (CaMV-t) was used to assess the bombardment parameters (FIG. 2). The plant expression vector was transformed into Escherichia coli (DH5α), followed by large-scale purification of plasmid DNA according to the manufacturer's instructions of the GenElute™ Plasmid Midiprep Kit (Sigma-Aldrich, SA). The circular plasmid DNA concentrations were adjusted to 1 μg/μl and used in all downstream applications. Assessment of DNA format (e.g. minimal cassette and whole plasmid), for efficient transient expression was evaluated. The minimal cassette was prepared with two restriction enzymes, i.e. HindIII and KpnI, to remove the vector backbone, resulting in a fragment containing only the ubiquitin promoter, reporter gene (gfp) and nos terminator. The digest was separated on a 1% agarose gel, and the appropriate fragment (3015 bp) was isolated and purified with the GeneJET Gel Extraction Kit according to manufacture protocol (Thermo Scientific, Inqaba, South Africa).

Particle Bombardment

Bombardment was done as described by Indra et al. (2006) with modifications. Briefly, all DNA deliveries were done via a locally engineered particle inflow gun. Tungsten particles (M10 Bio-Rad, South Africa) were sterilized with absolute ethanol and washed 3 times and re-suspended in sterile water, followed by the simultaneously addition of 2.4 M CaCl₂ and 0.1 M spermidine, vortexed and placed on ice. The aliquots of tungsten suspension were vortexed with 1 μg/ul concentrations of DNA. Incubation occurred at 4° C., followed by the removal of 100 μl of the aqueous layer prior to bombardment. For the bombardment, explants were clustered together (still exposed on turgidity treatment (osmoticum)), and placed 13 cm below the particle expelling tip, covering a 2 cm diameter circle and enclosed with a metal grid. The particle tip containing 1 mm² metal grid was loaded with 5 μl of the DNA suspensions. Air within the chamber was relinquished, until 80 kPa and 90 kPa, and the suspensions (DNA) were expelled when helium (1000 kPa) was released by a timer relay (0.05 s) 16 hours after bombardment, and the current media (turgidity treatment) was replaced with INDUC A media and maintained at 24° C. in the dark for 3-4 weeks.

Regeneration and Selection

At 5 weeks, embryogenic material was transferred to regeneration media for shoot formation, under a photoperiod of 16 h/8 h (day/night) at 24° C. This was also the selection phase, as the regeneration media (RDZA) also contained the antibiotic geneticin (45 mg/l) (note that selection started in either the first or second round of regeneration and continued in subsequent rounds). The selection phased lasted for 4-5 weeks. Plantlets that did not have adventitious roots were transferred to magentas containing either half or full strength MS media (Murashige and Skoog, 1962) until roots formed.

Ex Vitro Acclimatization

In vitro generated plantlets were hardened off in closed glass or plastic containers in a greenhouse at 28° C. Over time the containers were partially opened to allow acclimatization into the bigger, drier greenhouse environment. Plantlets were then regularly irrigated to ensure moist soil conditions.

Transient Expression Using GFP

Three to four days after bombardment, explants were assessed for transient GFP expression using a florescence microscope with special GFP filters, to eliminate the auto florescence. The fluorescence microscope was connected to an imaging system (Leica DC 200) to capture the image. The number of total cells per explant per plate, permitting the expression, was counted.

Results

Several experiments were conducted to assess four parameters that could lead to efficient transient gfp expression of immature embryos after bombardment. These parameters included the DNA format, concentration and pressure settings used during bombardment, and the osmoticum treatments of tissue pre and post-bombardment. A single wheat cultivar, Gamtoos S, was chosen due to its superior in vitro regeneration capabilities for all bombardment experiments. Whole plasmid (WP) and minimal cassette (MC) were assessed in combination with other parameters as listed in Table 1. The highest gfp expression was observed when pGFP was kept as a circular plasmid. The weakest transient expression was seen when pGFP was linearized.

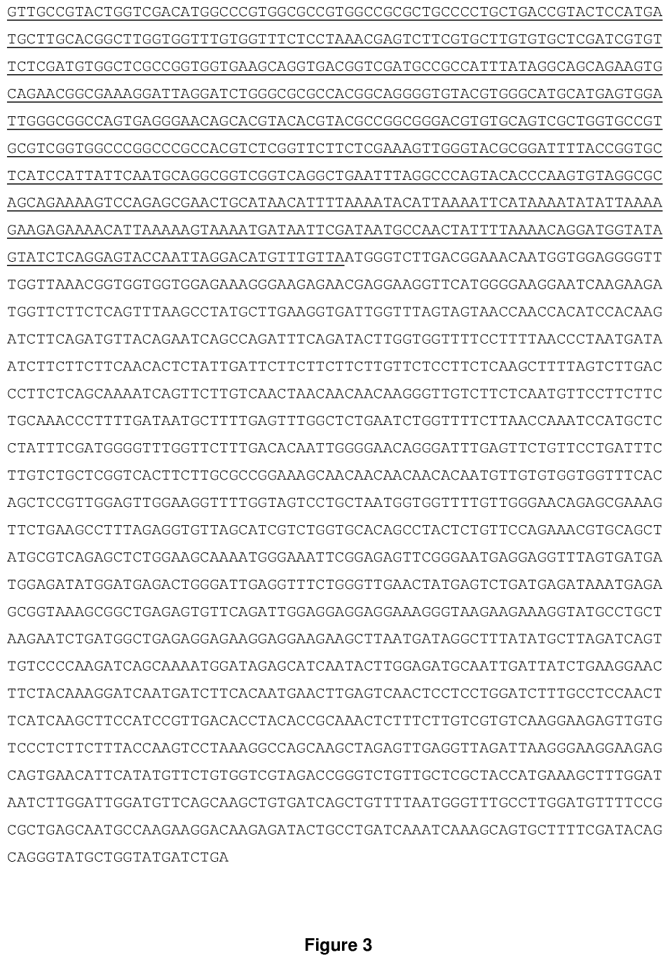

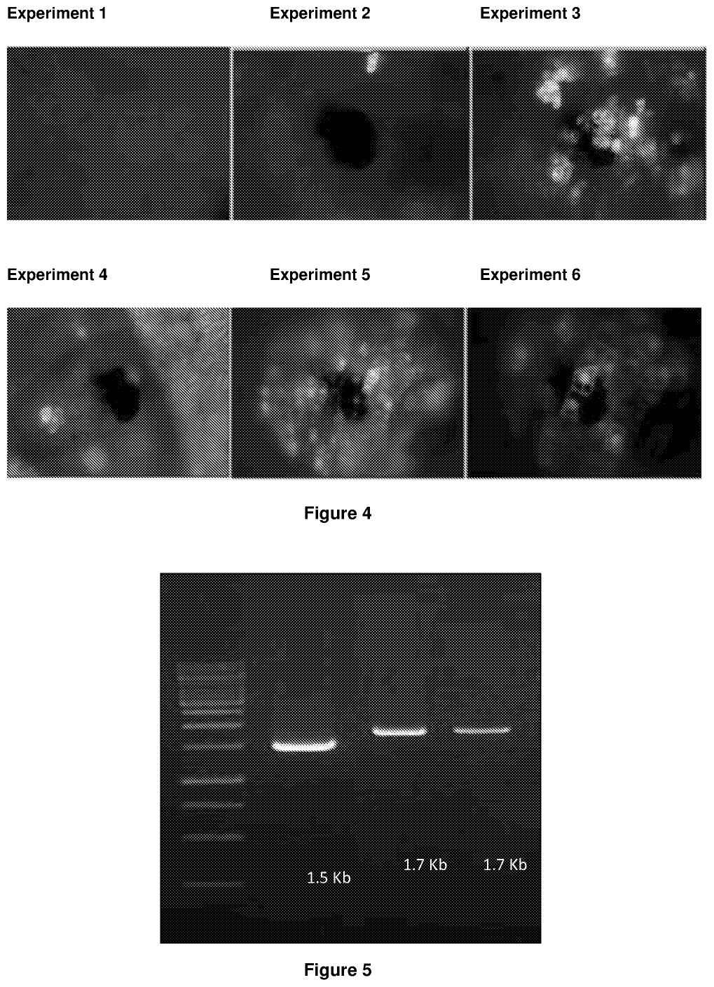

MC (0.5-1 μg/μl) in combination with WP (0.5 μg/μl-3 μg/μl) was not as effective as WP (5 μg/μl) on its own. WP (5 μg/μl) resulted in a transformation efficiency of between 43.3% and 46.6%, with an osmoticum treatment lasting maximally for 16 hours, and helium pressure at 80 kPa (Table 1; FIGS. 3 and 4). Although only transient GFP expression was assessed, these results served as the foundation in subsequent experiments for stable gene transfer and expression.

TABLE 1
Factors affecting the bombardment of 2-6 day old immature embryos, for
transient GFP expression
Induction A

| | pGFP | | pEmuKN | | Embryos | | | | | | |
|---|---|---|---|---|---|---|---|---|---|---|---|
| | (μg/ul) | | (μg/ul) | | amount | | Osmoticum | Total embryos | GFP foci** | | |
| Experiment | Linear | Circular | Circular | Pressure (kPa) | 1 mm | 3 mm | duration (hours) | with GFP | 1 mm | 3 mm | TTE |
| 1 | 1 | | 3 | 90 | 30 | 30 | 3 | — | — | — | 0% |
| 2 | — | 1 | 1 | 80 | 73 | — | 4 | 1 | 11 | | 1.37% |
| 3 | — | 5 | 5 | 80 | 30 | 30 | 16 | 14 | 229 | — | 46.6% |
| 4 | 0.5 | — | 0.5 | 90 | 30 | 30 | 16 | 1 | 1 | — | 3.3% |
| 5 | 1 | — | 3 | 80 | 30 | 30 | 3 | 7* | 10 | — | 23.3% |
| 6 | — | 5 | 5 | 90 | 30 | 30 | 16 | 13 | 149 | — | 43.3% |
| 7 | 0.5 | — | 0.5 | 80 | 30 | 30 | 16 | 4* | 20 | 0 | 13.3% |

**There is the possibility for human error during counting the foci, thus transient-transformation efficiency (TTE) is an approximation

Discussion

Particle bombardment is the most widely used method for transformation of cereal crops (Southgate et al., 1995; Rasco-Gaunt et al., 1999; Harwood et al., 2000; Rivera et al., 2012). However, optimization of parameters is crucial to improve on the widely published 1% transformation efficiency rate of wheat (Li et al., 2012). Transient GFP expression in Gamtoos allowed for the effective optimization of bombardment variables in this particular study. Since there are many parameters that can influence the transformation efficiency of wheat, those that are believed to be the most influential were evaluated when optimizing bombardment parameters.

Distance of micro-carrier to explant and embryo size made no significant difference in the transient GFP expression profile. However, pressure exerted on explants when delivering DNA had a significant difference, e.g. with the higher pressure (90 kPa) showing less GFP expression (149 foci). This might be due to tissue/cell damage (Folling, 2001). Using a pressure of 80 kPa resulted in much higher GFP expression levels (229 foci). This finding is supported by reports from Rasco-Gaunt et al. (1999). Low pressure is associated with uniform distribution of GFP expression, with substantial reduction in tissue shock and damage (Finer and McMullen, 1991).

Furthermore, DNA format, i.e. the use of minimal cassettes (MC), is often a requirement for commercialized GM products (Vianna et al., 2011). Multiple studies support the use of minimal cassettes to obtain transgenic monocot plants such as rice (Loc et al, 2002). However, with the investigation of DNA format, the results show that the use of a MC resulted in less transient GFP expression (0%-23.3%) in contrast to WP (1.3%-46.6%). DNA format is also closely linked to the DNA concentration used for bombardment (Jackson et al., 2013). 5 μg/μl of circular DNA resulted in the highest transient gfp expression (Table 1; FIGS. 3 and 4).

The final parameter assessed was the osmoticum treatment after bombardment. This is crucial since it minimises cytoplasm leakage from target cells, preventing the loss of DNA that has penetrated the bombarded cells. The results show that post-conditioning of explants for a maximum of 16 hours on non-metabolisable osmotica such as mannitol and sorbitol leads to effective plasmolysis, which is also supported by Indra et al. (2006). Although only transient gfp expression was evaluated, the purpose of this particular study was not to obtain stable transgenic plants.

Transformation Using SUMO Targets

Gene Identification and Vector Construction

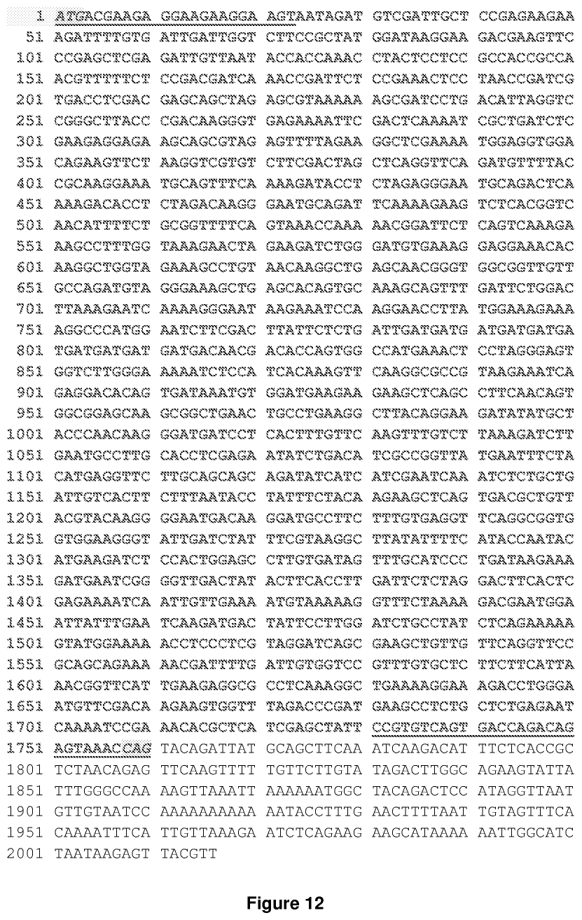

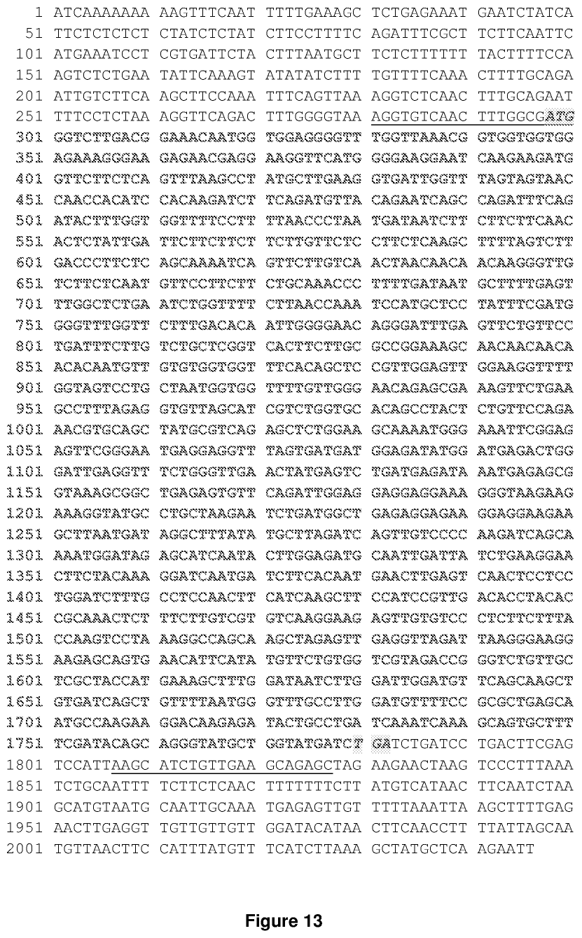

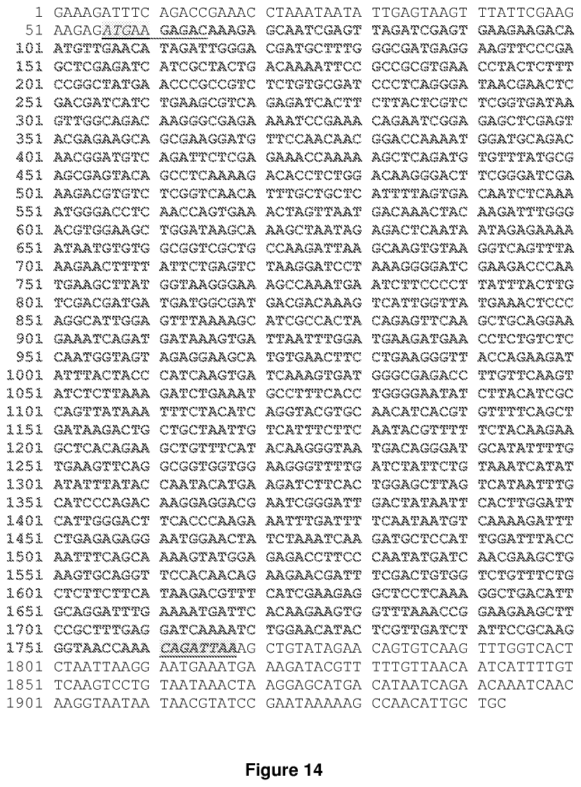

Arabidopsis thaliana seeds were growth in a growth chamber under controlled humidity and light. At 4 weeks leaf material was harvested and total RNA isolated and quantified (ng/μl). RNA was then used as template for cDNA synthesis using First-strand cDNA Synthesis Kit, and the integrity of the cNDA was confirmed by amplifying a house-keeping gene known as actin. Bioinformatics tools were applied to receive information regarding the gene ontology (OTS1:AT1G60220 (SEQ ID NO. 4, FIG. 12), OTS2:AT1G10570 (SEQ ID NO. 6, FIG. 14) and ICE1: AT3G26744.4 (SEQ ID NO. 5, FIG. 13), which was used during primer design. All primers were diluted according to manufacturer's recommendation (lnqaba Biotec). With extensive optimization all three genes were amplified using Phusion High-Fidelity DNA Polymerase (Thermo Scientific). Gel electrophoresis confirmed the correct size fragments (FIG. 5), which were then excised using a

GenElute Gel Extraction Kit and quantified (ng/μl). Fragments were independently cloned into a storing vector known as pJET1.2/blunt and heat-shock transformed into E. coli DH5α, followed by a colony PCR using the gene specific primers to confirm gene presence. GenElute Plasmid Miniprep Kit was done on positive clones followed by sequencing at a local sequencing unit (CAF) which confirmed 100% identity to the corresponding genes on “The Arabidopsis Information Resource (TAIR)” database.

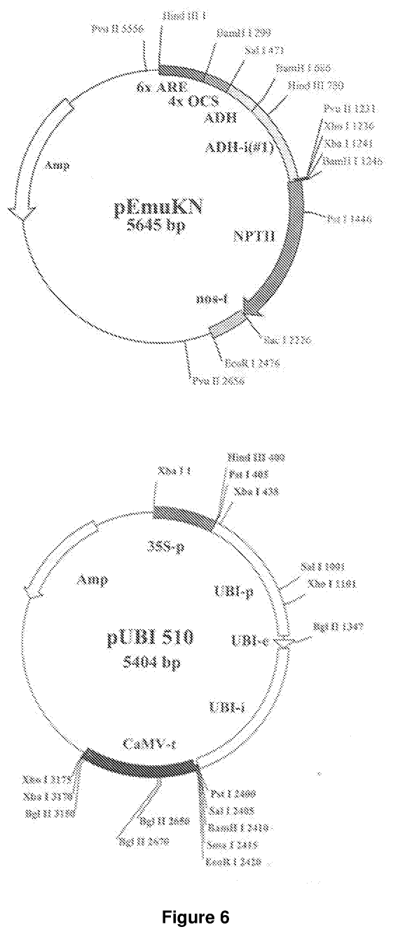

Since nBLAST results confirmed that OTS1, OTS2 and ICE1 had indeed been amplified and isolated, these genes were excised (double enzymatic digest) from storage vectors (pJect::ots1, pjet::ots2 and pjet::ICE), blunted and phosphorylated for ligation into a prepared plant expression vector, known as pUbi510, under the control of a well characterized constitutive ubiquitin promoter and CamV-t terminator (FIG. 6). The plant expression vector required further preparation, which entailed a single blunt-end digest (Smal) to linearize the vector followed by dephosphorylation. After successfully ligating the genes independently into pUbi510, each construct was heat-shock transformed into E. coli DH5α, followed by a colony PCR to confirm the 5′-3′ orientation of each gene. Again, sequencing confirmed the aforementioned. This resulted in three constitutive constructs known as a) Pubi510::OTS1, b) Pubi510::OTS2, and c) Pubi510::ICE1.

According to literature regarding ICE1, constitutive expression of this gene could potentially result in a metabolically skewed plant with severely retarded growth. This is of particular concern in the context of the present study as this could affect the yield of wheat and other cereals or grasses. However, this has not been confirmed in a hexaploid species such as wheat. It was hypothesised that placing ICE1 in a vector under the control of a drought inducible promoter could not only avoid yield penalty, but increase the plant abiotic fitness (drought, heat, cold, etc). The promoter Rab17 (Accession: x15994.1) from maize was selected, as data supports that this drought inducible promoter contains all necessary cis-elements and is not leaky in hexaploids.

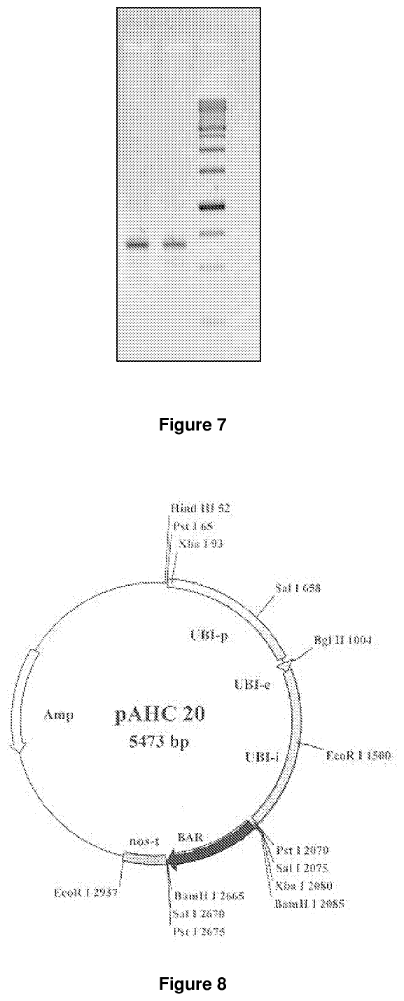

Bioinformatics tools were again applied, where the entire gene (1989 bp) was examined, gDNA was isolated from four week old maize callus, followed by High-Fidelity nested amplification of the Rab17 promoter region (45 . . . −654 bp)(FIG. 7), and again this was cloned into a storage vector for glycerol stock and sequencing purposes. The identity (100%) of Rab17was confirmed on NCBI. The vector pAHC20 (FIG. 8) was then modified by removing its endogenous promoter (ubiquitin) by single restriction digest (XbaI), blunting and dephosphorylating, and replacing it with the phosphorylated Rab17. The construct was transformed into E. coli followed by colony PCR, which confirmed pAHC20::Rab17 and its 5′-3′ orientation. This plasmid was further manipulated by removing its reporter gene (Bar) through double digestion (BamH I) and dephosphorylation. Finally the ICE1 gene was phosphorylated and ligation set up overnight (pAHC20::Rab17::ICE).

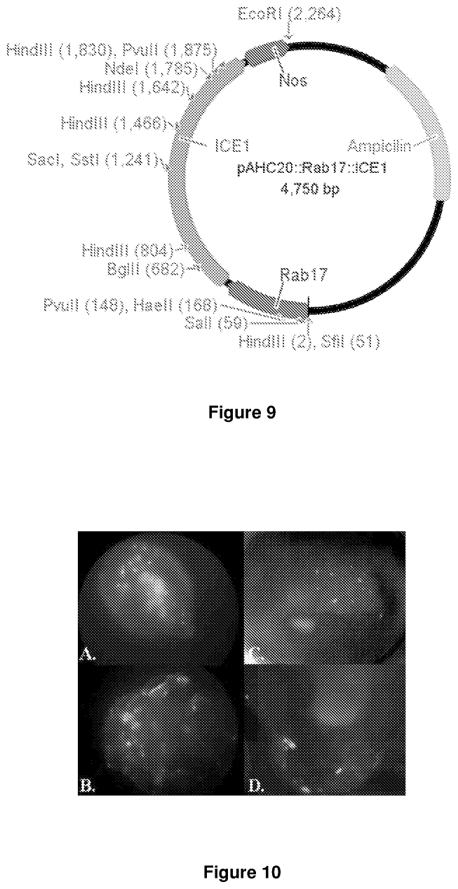

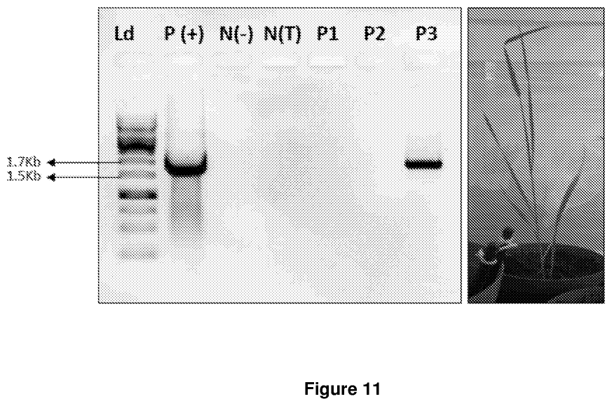

After successful ligation, the construct was heat-shock transformed into E. coli DH5α, followed by a colony PCR to confirm RAB17:ICE 5′-3′ orientation in pAHC20 (FIGS. 9 and 10). Sequencing confirmed the aforementioned orientation.

Six vectors (Pubi510:OTS1, Pubi510:0TS2, Pubi510:ICE1, pAHC20:RAB17:ICE1, Pubi510 (empty-control) and pEmuKN) were transformed into Escherichia coli (DH5α) followed by large-scale purification according to the manufacturer's instructions of the GenElute™ Plasmid Midiprep Kit (Sigma-Aldrich, SA). Note that pEmuKN is a selection vector which houses the selection gene known as nptII. All plasmid DNA concentrations were adjusted to 1 μg/μl (the aforementioned concentration was determined as describer earlier herein).

Characterization of the Transgene and Modified Plant

Verifying Stable Expression in Transformants and Progeny

Plants were firstly assessed through amplification of the sequences of the vector boundaries of the inserted constructs using PCR. If PCR amplicons were produced (and thus sequences are present in the genomic DNA of the samples), then an insertion event was confirmed. Secondly, the expression of the gene inserted into the tissue was quantified using RT-qPCR. Again, primer sequences specific to the gene of interest were applied. However, in this instance, RNA was extracted and cDNA (complementary-DNA) was synthesized, which were then used as template for the amplification of products.

Transgene Confirmation

T₀ generation plants were analyzed for the presence of transgene using specific primers. Genomic DNA was isolated from fresh 4 leaf stage transformed and non-transformed (NT) plants using Fermentas Gene JET Plant Genomic DNA Purification Mini Kit (#k0791) according to the manufacturer's instructions. Each PCR reaction had approximately 150 ng of the genomic DNA template, 10 mM of dNTP, 10 μl of 5× Green GoTaq buffer reaction buffer® (containing 1.5 mM MgCl₂), GoTaq® DNA Polymerase (5 μ/ul). One primer was promoter-specific (Ubi-exp: 5′-ATACGCTATTTATTTGCTTGG-′3) (SEQ ID NO. 7) and the second primer gene-specific 5′-CTGGTTTACTCTGTCTGGTCACT-3′ (OTS1-REV) (SEQ ID NO. 8). PCR was initiated by denaturation at 95° C., for 3 min followed by 35 cycles of 1 min at 95° C. (denaturation), 30s at 60° C. (annealing) and 72° C. for 2 min (extension), ending off with 1 cycles of 72° C. for 10 min (final extension). Sample was loaded with appropriate controls and was subjected to electrophoresis on 1% Agrose gel at 90V/aM for 40 min.

Enzymatic Activity: Assaying Reactive Oxygen Species (ROS) and Pathogenesis Related (PR) Protein Activity

To verify the potential biotic and abiotic stress tolerance, selected marker protein activities (i.e., peroxidise, GST and β-1,3-glucanase) were assayed in some of the transgenic plants. The T₁ generation plants were assayed for ROS and PR protein activities using the method as previously described by Botha et al. (2014).

Results

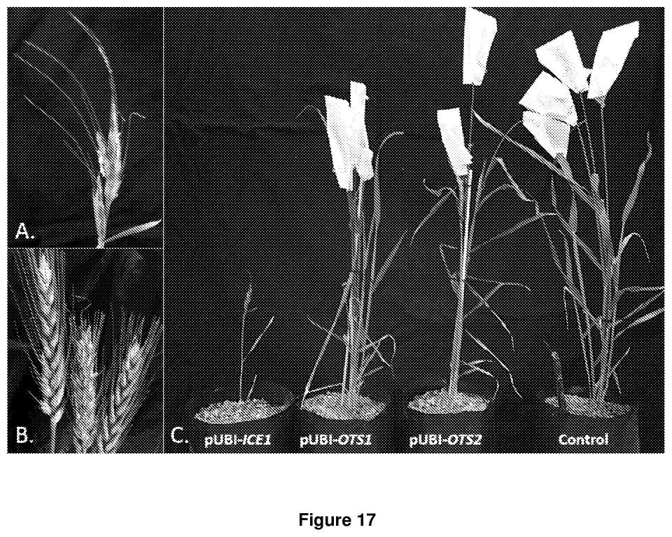

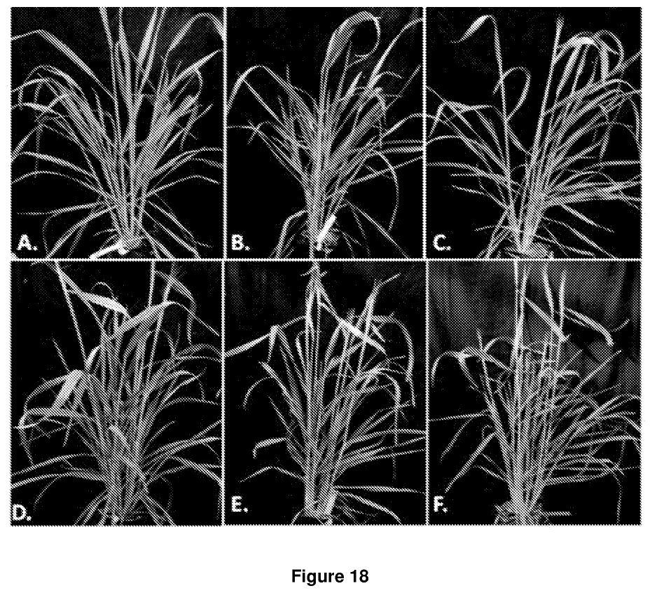

DNA was extracted from T₁ generation plants and genomic DNA used as template to confirm transgene insertion in the putative transgenic plants. Five plants tested positive for pUBI:OTS1 with fragment size of 1700 bp, two plants for pUBI:OTS2 with fragment size of 1700 bp, and one for pUBI:ICE1 with fragment size of 1500 bp (one pUBI:OTS1 plant died) (FIGS. 16 to 18). The transgenic plants pUBI:OTS1 and pUBI:OTS2 plants displayed normal phenotypes. However, the transgenic pUBI:ICE1 plant displayed stunted growth (FIGS. 17 and 18).

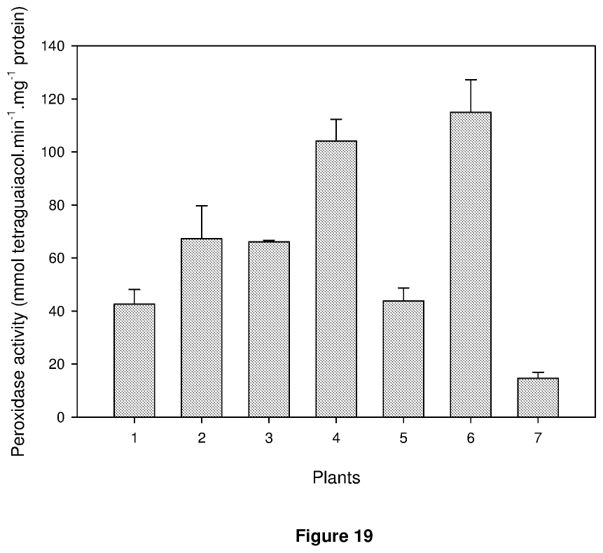

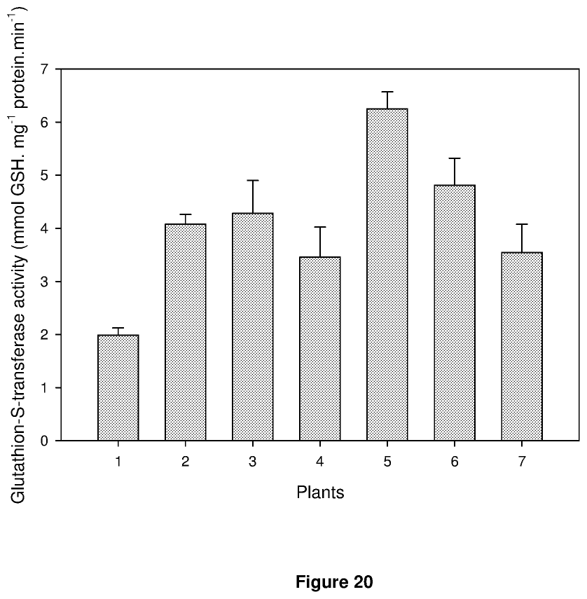

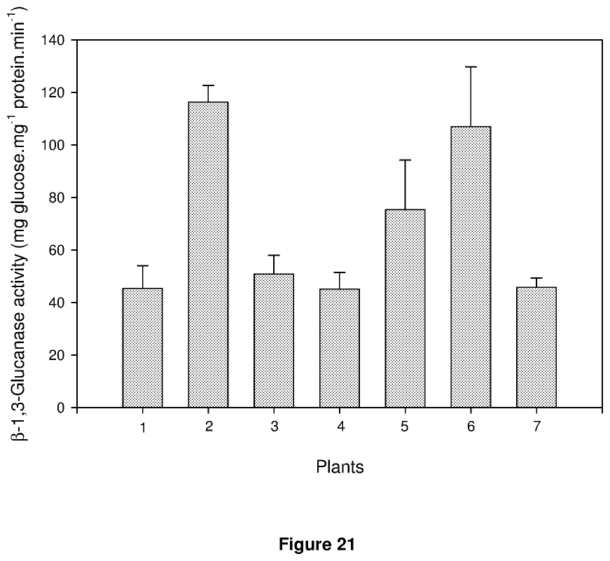

Functional characterisation of these plants revealed enhanced increased expression of reactive oxygen species (Le., peroxidase and GTS activity) and pathogenesis related (PR, i.e., β-1, 3-1,3-glucanase) protein activity (FIGS. 19-21) in transgenic plants when compared to untransformed control wheat plants, suggesting increased abiotic and biotic stress tolerance in these transgenic plants.

### EXAMPLE 2

### Transformation of Sugarcane to Enhance Stress Tolerance

Sugarcane In Vitro Regeneration

Materials and Methods

Plasmids

Gene inserts (as described above) were cloned into the multiple cloning site of plant expression vector, pUbi510 (FIG. 6). This vector contained an ubiquitin promoter and a 35S cauliflower mosaic virus terminator sequence. A second 5.6 kB size plasmid, pEmuKN, contained the nptII coding sequence driven by the strong monocot Emu promoter sequence, and nos terminator (FIG. 6), allowing efficient selection on geneticin (Last et al., 1990).

Explant Material and Culture Conditions

In order to produce embryonic callus, the basal part of sugarcane (Saccharum spp. hybrids) cultivar NCo310 leaf roll, just above the apical meristem, was excised, cut into 2 mm thick transverse sections and placed on MS medium (Murashige and Skoog, 1962; Highveld Biological, South Africa [SA]) containing 2% (w/v) sucrose, 3 mg/I 2,4-D, 0.5 g casein, and 0.22% (w/v) gelrite (Sigma-Aldrich, SA), pH 6.0 (MS3). Cultures were incubated for 6 to 8 weeks at 24° C. in the dark and placed on fresh medium every two weeks.

Transformation via Particle Bombardment

Materials and Methods

Plant Transformation

A particle inflow gun, constructed locally, was used for all DNA deliveries. Bombardment was done as described by Franks and Birch (1991) with modifications. Tungsten particles (5 mg; M10; Biorad, Calif.) were sterilized with 100% ethanol, vortex and rinsed three times with sterile water and re-suspended in 50 μl sterile water. The tungsten suspension was mixed with 10 μl plasmid DNA (5 μg of each plasmid), 50 μl 2.5 M CaCl₂ and 20 μl M spermidine. The mixture was incubated on ice and 100 μl of the aqueous layer removed prior to plant explant bombardment. For bombardment, 5 μl of the particle suspension was placed in the centre of a 1 mm² metal grid above the explants. Target tissue was placed 16.5 cm below the particle source and covered with a metal grid. The chamber was set under a vacuum of 80 kPa and the particles were discharged when helium, 1000 kPa, was released by a timer relay, 0.05 sec.

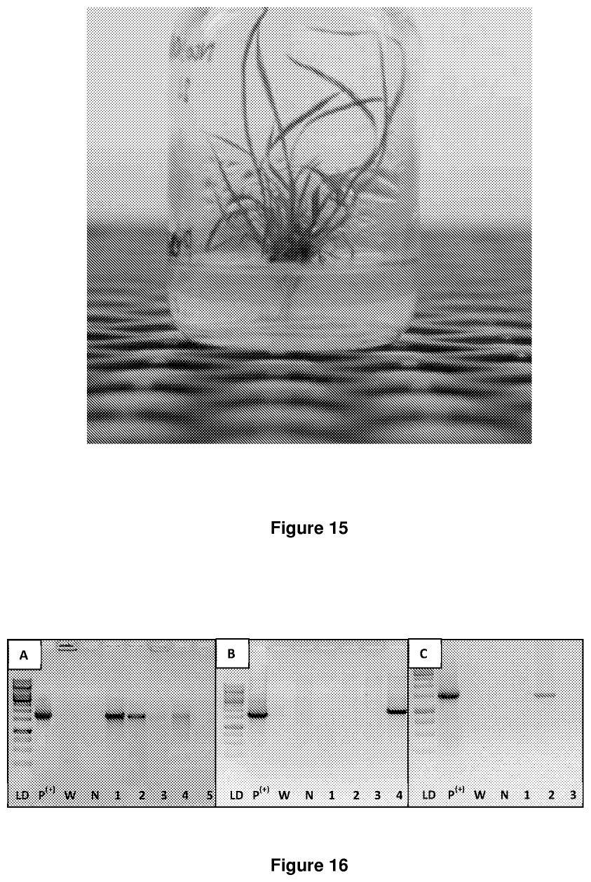

Harvested embryogenic sugarcane calli clumps (on average 3-4 mm diameter a piece) were stacked closely together in a circle, with a size of 3 cm diameter, on osmoticum medium consisting of the basal MS3 medium with 0.2 M each of sorbitol and mannitol 4 h prior to and after bombardment (Vain and McMullen, 1993). After bombardment, the plant tissue was maintained on MS3 medium for 3 days before selection started on 45 mg/l geneticin (Sigma, UK) for 8-10 weeks in the dark as outlined by Bower and Birch (1992). On average ten plates of embryogenic callus circle portions were bombarded for each transgene. After selection, newly regenerated calli, surviving selection, were placed on MSO medium, lacking 2.4-D for 8 weeks until roots has formed. Regeneration was done in a growth room at 26° C. in a 16/8 light/dark regime. Plantlets, 5 cm high with roots (FIG. 15), were hardened off in a mix of potting soil, sand and vermiculite at a ratio of 2:1:1 in pots in the glasshouse.

### REFERENCES

Abdul R, Ma Z, Wang H. 2010. Genetic transformation of wheat (Titicum aestivum L): A review. Triticeae Genomics and Genetics 1(2): 1-7. DOI: 10.5376/tgg.2010.01.0002
Barro F, Martin A, Lazzeri P A and Barcelo P. 1999. Medium optimization for efficient somatic embryogenesis and plant regeneration from immature inflorescences and immature scutella of elite cultivars of wheat, barley and tritordeum. Euphytica 108: 161-167.
Benson E E. 2008. Cryopreservation of phytodiversity: A critical appraisal of theory and practice. Critical Reviews in Plant Sciences 27 (3): 141-219. doi:10.1080/07352680802202034
Bhalla P L, Ottenhof H H, Singh M B. 2006. Wheat transformation—an update of recent progress. Euphytica 149: 353-366.
Biddington N L. 1992. The influence of ethylene in plant tissue culture. Plant Growth Regulation 11: 173-178.
Bleecker A B and Kende H. 2000. Ethylene: A gaseous signal molecule in plants. Annual Review of Cell and Developmental Biology 16:1-18.
Botha A-M, Van Eck L, Burger N F V, Swanevelder Z H. 2014. Near-isogenic lines of Triticum aestivum with distinct modes of resistance exhibit dissimilar transcriptional regulation during Diuraphis noxia feeding. Biology Open 3: 1116-1126.
Bower R, Birch R. 1992. Transgenic sugarcane plants via micro-projectile bombardment. Plant J 2:409-416
Bian H W, Wang J H, Lin W Q, Han N, Zhu M Y. 2002. Accumulation of soluble sugars, heat-stable proteins and dehydrins in cryopreservation of protocorm-like bodies of Dendrobium candidum by the air-drying method. Journal of Plant Physiology 159:1139-1145

Chen T H H, Kartha K K, Gusta L V. 1985. Cryopreservation of wheat suspension culture and regenerable callus. Plant Cell, Tissue and Organ Culture 4(2): 101-109.
Chugh A, Vikrant, Mahalakshmi A, Khurana P. 2012. A novel approach for Agrobacterium-mediate germ line transformation of Indian bread wheat (Triticum aestivum) and pasta wheat (Triticum durum). Journal of Phytology 4(2): 22-29.
Coleman A. 2014. Farming for tomorrow—The Russian wheat aphid. Farmer's weekly, South Africa p 22.
Du Toit and Walters. 1984. Damage assessment and economic threshold values for the chemical control of the Tussiona wheat aphid, Diuraphis noxia (Moldvilko), on winter wheat. Technical Communication, Department of Agriculture, RSA 191: 58-62.Engelmann F. 2004. Plant cryopreservation: Progress and prospects. In Vitro Cell Dev Biol—Plant 40:427-433
Engelmann F. 2011. Cryopreservation of embryos: An overview. In: Thorpe T A, Yeung E C (eds), Plant Embryo Culture: Methods and Protocols, Methods in Molecular Biology 710: 155-184. Springer Science and Business Media, LLC. doi: 10.1007/978-1-61737-988-8_13
Feuillet C, Langridge P, Waugh R. 2007. Cereal breeding takes a walk on the wild side. Trends in Genetics 24: 24-32
Finer J, McMullen M D. 1991. Transformation of soybean via particle bombardment of embryogenic. In vitro Cellular & Developmental Biology—Plant 27: 175-182
Folling L O. 2001. Transformation of wheat (Triticum aestivum L.) microspore-derived callus and microspores by particle bombardment. Plant Cell Reports 20: 629-636
Food and Agriculture Organization of the United Nations. 2013. FAOSTAT. http://faostat.fao.org
Franks T, Birch R G. 1991. Gene transfer into intact sugarcane callus using microprojectile bombardment. Aus J Plant Physiol 18:471-480

Fretz A and Lorz H. 1995. Cryopreservation of in vitro cultures of barley (Hordeum vulgare L. and H. murinum L.) and transgenic cells of wheat (Triticum aestivum L.). Journal of Plant Physiology 146: 489-496.
Gamborg O L, Miller R A, Ojima K. 1968. Nutrient requirements of suspension culture of soybean root cells. Exp. Cell Res 50: 151-158.
Gil-Humanes J, Ozuna C V, Marin S, León E, Barro F, Pist\acute{0}n F. 2011. Genetic Transformation of Wheat: Advances in the Transformation Method and Applications for Obtaining Lines with Improved Bread-Making Quality and Low Toxicity in Relation to Celiac Disease, Genetic Transformation, ISBN: 978-953-307-364-4. InTech. http://www.intechopen.com/books/genetic-transformation/genetic-transformation-of-wheat-advances-in-thetransformation-method-and-applications-for-obtaining.

Harwood W A, Ross S M, Cilento P, Snape J W, Centre JI, Nr N. 2000. The effect of DNA/gold particle preparation technique, and particle bombardment device, on the transformation of barley (Hordeum vulgare). Euphytica 111: 67-76
Harding K. 2004. Genetic integrity of cryopreserved plant cells: A review. Cryo Lett 25:3-22
He Y, Jones H D, Chen S, Chen X M, Wang W D, Li K X, Wang D S, Xia LQ. 2010. Agrobacterium-mediated transformation of durum wheat (Triticum turgidum L. var. durum cv Stewart) with improved efficiency. Journal of Experimental Botany 61 (6): 1567-1581
Hoekstra A F, Golovina A E, Buitink J. 2001. Mechanisms of plant desiccation tolerance. Trends Plant Sci 6:431-438
Hyde C L and Phillips G C. 1996. Silver nitrate promotes shoot development and plant regeneration of chile pepper (Capsicum annuum L.) via organogenesis. In Vitro Cell and Developmental Biology 32: 72-80.
Indra K, Vasil V, Vimla V. 2006. Loyola-Vargas F, Victor M, Vázquez-Flota II. Plant Cell Culture Protocols: Methods in Molecular Biology 1(2): 273-283. Humana Press Inc. Totowa, N.J.
Jackson M A, Anderson D J, Birch R G. 2013. Comparison of Agrobacterium and particle bombardment using whole plasmid or minimal cassette for production of high-expressing, low-copy transgenic plants. Transgenic Research 22: 143-51
Jahne A, Lazzeri P A, Jager-Gussen M, Lorz H. 1991. Plant regeneration from embryogenic cell suspensions derived from anther cultures of barley (Hordeum vulgare L.). Theoretical and Applied Genetics 82: 74-80.
Jitsopakul N,Thammasiri K, Ishikawa K. 2008. Cryopreservation of Bletilla striata mature seeds, 3-day germinating seeds and protocorms by droplet-vitrification. Cryoletters 29 (6): 517-526
Jones H D, Dohery A, Wu H. 2005. Review of methodologies and a protocol for the Agrobacterium-mediated transformation of wheat. Plant Methods 1: 5. DOI: 10.1186/1746-4811-1-5.
Kavas M, Öktem H A, Yücel M. 2008. Factors affecting plant regeneration from immature inflorescence of two winter wheat cultivars. Biologia Plantarum 52: 621-626.
Kendal E J, Kartha K K, Qureshi J A and Chermak P. 1993. Cryopreservation of immature spring wheat zygotic embryos using an abscisic acid pretreatment. Plant Cell Reports 12(2): 88-94.
Kendal E J, Qureshi J A, Kartha K K, Leung N, Chevrier N, Caswell K and Chen T H H. 1990. Regeneration of freezing-tolerant spring wheat (Triticum aestivum L.) plants from cryoselected callus. Plant Physiology 94: 1756-1762.
Khoddamzadeh A A, Sinnah U R, Lynch P, Kadir M A, Kadzimin S B, Mahmood M. 2011. Cryopreservation of protocorm-like bodies (PLBs) of Phalaenopsis bellina (Rchb.f.) christenson by encapsulation dehydration. Plant Cell Tiss Organ Cult 107: 471-481. doi: 10.1007/s11240-011-9997-4.
Kumar V, Parvatam G and Ravishankar G A. 2009. AgNO3—a potential regulator of ethylene activity and plant growth modulator. Electronic Journal of Biotechnology 12(2): 1-15.
Lacock L, Botha A-M. 2000. Genotype variation in regeneration and transient expression efficiencies of 14 South African wheat cultivars. S. Afr. Tydskr. Plant Grond 17(4): 170-174.
Lacock L. 1999. Genotype variation in regeneration and transformation efficiencies of South African wheat cultivars. MSc thesis, University of Pretoria, South Africa.
Lambardi M, Benelli C, De Carlo A. 2005. Cryopreservation as a tool for the long-term conservation of woody plant germplasm: Development of the technology at the CNR/IVALSA Institute of Florence. The Role of Biotechnology, Turin, Italy, pp181-182. http://www.fao.org/biotech/docs/lambardi.pdf
Last D I, Bretell R I S, Chamberlain D A, Chaudhury A M, Larkin P J, Marsh E L, Peacock W J, Dennis E S. 1990. pEmu: an improved vector for gene expression in cereal cells. Theoretical and Applied Genetics 81: 581-588.
Li J, Ye X, An B, Du L, Xu H. 2012. Genetic transformation of wheat: current status and future prospects. Plant Biotechnology Reports 6: 183-193glycol)-poly(lactic acid) nanoparticles for intranasal delivery to the brain. Toxicology and applied pharmacology 251: 79-84
Liao X, Tang L and Zhang F. 2005. Effect of silver nitrate on callus regeneration and activity of some enzymes during tissue culture of wheat immature inflorescence. http://www.paper.edu.cn.
Loc N T, Tinjuangjun P, Gatehouse A M R, Christou P, Gatehouse J A. 2002. Linear transgene constructs lacking vector backbone sequences generate transgenic rice plants which accumulate higher levels of proteins conferring insect resistance. Molecular Breeding 9: 231-244
Marasas C N, Anandajayasekeram P, Millard S, van Rooyen C J. 2005. Farm-level adoption and impact of agricultural technology: the case of Russian wheat aphid resistant cultivars in South Africa. Sout African J of Agric Ext (S. Afr Tydskr Landbouvoorl) 34: 318-333.
Miroshnichenko D N, Filippov M V, Dolgov S V. 2013. Medium optimization for efficient somatic embryogenesis and in vitro plant regeneration of spring common wheat varieties. Russian Agricultural Sciences 39: 24-28.
Moghaieb REA, El-Arabi N I, Momtaz O A, Youssef S S, Soliman M H. 2010. Genetic transformation of mature embryos of bread (T. aestivum) and pasta (T. durum) wheat genotypes. GM Crops 1 (2): 87-93.
Moran M, Cacho M, Fernandez-Tarrago J, Corchete P. 1999. A protocol for the cryopreservation of Digitalis thapsi L. cell cultures. Cryo Letters 20: 193-198
Murashige T and Skoog F. 1962. A revised medium for rapid growth and bio-assays with tobacco tissue cutures. Physiol Plant 15: 473-497.
Ogawa Y, Sakurai N, Oikawa A, Kai K, Morishita Y, MoriK, Moriya K, Fujii F, Aoki K, Suzuki H, Ohta D, Saito K, Shibata D. 2012. High-throughput cryopreservation of plant cell cultures for functional genomics. Plant Cell Physiol 53 (5): 943-952. doi: 10.1093/pcp/pcs038
Ponya Z and Barnabas B. 2003. Regeneration of fertile wheat (Triticum aestivum) plants from isolated zygotes using wheat microspore culture as nurse cells. Plant Cell, Tissue and Organ Culture 74: 243-247.
Popelka J C, Gollasch S, Moore A, Molvig L, Higgins T J V. 2006. Genetic transformation of cowpea (Vigna unguiculata L.) and stable transmission of the transgenes to progeny. Plant Cell Rep 25: 304-312
Popov S A, Popova V E, Nikishina V T, Vysotskaya N O. 2006. Cryobank of plant genetic resources in Russian Academy of Sciences. Int J Refrig 29:403-410
Rasco-Gaunt S, Riley a, Barcelo P, Lazzeri P A. 1999. Analysis of particle bombardment parameters to optimise DNA delivery into wheat tissues. Plant Cell Reports 19: 118-127
Rasco-Gaunt S, Riley A, Cannell M, Barcelo P and Lazzeri PA. 2001. Procedures allowing the transformation of a range of European elite wheat (Triticum aestivum L.) varieties via particle bombardment. Journal of Experimental Botany 50 (357): 865-874.
Razdan M K. Introduction to plant tissue culture. Science Publishers Inc, New Hampshire, USA, pp 26.
Rivera A L, Gómez-Lim M, Fernández F, Loske A M. 2012. Physical methods for genetic plant transformation. Physics of Life Reviews 9: 308-45
Samygin G A. 1994. Causes of damage of plant cells by extracellular ice. Fiziol Rast 41: 614-625.
She M, Yin G, Li J, Li X, Du L, Ma W, Ye X. 2013. Efficient regeneration potential is closely related to auxin exposure time and catalase metabolism during the somatic embryogenesis of immature embryos in Triticum aestivum L. Molecular Biotechnology 54: 451-60.
Solov'eva A I, Vysotskaya O N, Popov A S and Dolgikh Y l. 2010. Freezing of dehydrated calli of spring wheat (Triticum aestivum L.) in liquid nitrogen and their morphogenetic potential. Biology Bulletin 37(5): 489-495.
Southgate E, Davey M R, Power J B, Marchant R. 1995. Factors affecting the genetic engineering of plants by microprojectile bombardment. Biotechlology Advances 13: 631-651
Tester, M. and Bacic, A. 2005. Abiotic Stress Tolerance in Grasses. From Model Plants to Crop Plants. Plant Physiology 137: 797-793.
Tsukazaki H, Mii M, Tokuhara K, Ishikawa K. 2000. Cryopreservation of Doritaenopsis suspension culture by vitrification. Plant Cell Reports 19: 1160-1164
Vain P, Flament P and Soudain P. 1989. Role of ethylene in embryogenic callus initiation and regeneration in Zea mays L. Journal of Plant Physiology 146: 537-540.
Vain P, McMullen M D, Finer J J. 1993. Osmotic treatment enhances particle bombardment-mediated transient and stable transformation of maize. Plant Cell Rep 12:84-88
Van der Vyver A. 2013. The relative value between barley and wheat from a production point of view: Northern Cape irrigation areas. Department of Agricultural Economics, Extension and Rural Development, University of Pretoria. http://www.grainsa.co.za/the-relative-value-between-barley-and-wheat-from-a-production-point-of-view:-northern-cape-irriqation-areas
Vannini G L and Poli F. 1983. Binucleation and abnormal chromosome distribution in Euglena gracilis cells treated with dimethyl sulfoxide. Protoplasma 114: 62-66.
Vasil I K and Vasil V. 2006. Transformation of wheat via particle bombardment. In: Loyola-Vargas V M and Vazquez-Flota F. Plant Cell Culture Protocols, Methods in Molecular Biology 318: 273-283.
Vianna G R, Aragão F J L, Rech E L. 2011. A minimal DNA cassette as a vector for genetic transformation of soybean (Glycine max). Genetics and molecular research : GMR 10: 382-90
Viertel K and Hess D. 1996. Shoot tips of wheat as an alternative source for regenerable embryogenic callus cultures. Plant Cell, Tissue and Organ Culture 44: 183-188.
Walters M C. 1984. Progress in Russian wheat aphid (Diuraphis noxia Mordw.) research in the Republic of South Africa, In: Proceedings, meetings of the Russian Aphid Task Team, University of the Orange Free State, May 1982, Bloemfontein, South Africa.
Wang G, Wang Q, Zhang W, Yao D, Zheng W, Fan H, Wang H and Wang F. 2011. Effect of cryopreservation in liquid nitrogen on germination rate and vigor of wheat seeds. Chinese Seed 2(1): 1-4.
Wang X, Xian R, Gui-xiang Y, Wang K, Li J, Du L, Xu H and Ye X. 2013. Effects of environmental temperature on the regeneration frequency of the immature embryos of wheat (Triticum aestivum L.). Journal of Integrative Agriculture 1281
Wheat. 2009. Resource Centre, Department of Agriculture, Forestry and Fisheries, Pretoria, South Agrica.
Wu H, Sparks C, Amoah B, Jones H D. 2003. Factors influencing successful Agrobacterium-mediated genetic transformation of wheat. Plant Cell Reports 21: 659-668.
Wu L M, Wei Y M and Zheng Y L. 2006. Effects of silver nitrate on the tissue culture of immature wheat embryos. Russian Journal of Plant Physiology 53 (4): 530-534.
Yi H E, Qiong W, Jian Z, Tao SUN, Guang-xiao Y, Guang-yuan H E. 2015. Current status and trends of wheat genetic transformation studies in China. 14: 438-452
Yin G, Wang Y, She M, Du L, Xu H, Ma J, Ye X. 2011. Establishment of a highly efficient regeneration system for the mature embryo culture of wheat. Agricultural Sciences in China 10: 9-17.
Zhao T J, Zhao S Y, Chen H M, Zhao Q Z, Hu Z M, Hou BK, Xia G M. 2006. Transgenic wheat progeny resistant to powdery mildew generated by Agrobaterium inoculum to the basal portion of wheat seedling. Plant Cell Reports 25: 1199-1204.
Ziemienowicz A. 2013. Agrobacterium-mediated plant transformation: factors, applications and recent advances. Biocatalysis and Agricultural Biotechnology. doi: 10.1016/j.bcab.2013.10.004

1. A method for producing a transformed plant of the family Poaceae, the method comprising the step of introducing at least one nucleic acid encoding a gene selected from the group consisting of OTS1, OTS2 and ICE1 into the genome of the plant, wherein the nucleic acid has a sequence which is at least 80% identical to SEQ ID NO. 3, SEQ ID NO. 9 or SEQ ID NO. 11, respectively, and wherein transformed plant has enhanced tolerance to an abiotic stress compared to an untransformed plant.

2. A method according to claim 1, wherein the gene is under the control of a drought inducible promoter.

3. A method according to claim 2, wherein the promoter is the Rab17 promoter (SEQ ID NO. 2).

4. A method according to claim 1, wherein the plant is a cereal or grass.

5. A method according to claim 4, wherein cereal or grass is selected from the group consisting of wheat, barley, sorghum, maize, sugarcane, oats, rye, triticale and fodder grass species.

6. A method according to claim 5, wherein the plant is wheat.

7. A method according to claim 5, wherein the plant is sugarcane.

8. A method according to claim 1, wherein the abiotic stress is drought.

9. A method according to claim 1, wherein the abiotic stress is heat.

10. A method according to claim 1, wherein the abiotic stress is cold.

11. A method according to claim 1, wherein the abiotic stress is salinity.

12. A method according to claim 1, wherein the gene is OTS1 and the sequence of the introduced nucleic acid is at least 80% identical to SEQ ID NO. 9.

13. A method according to claim 12, wherein the sequence of the introduced nucleic acid is identical to SEQ ID NO. 9.

14. A method according to claim 1, wherein the gene is OTS2 and the sequence of the introduced nucleic acid is at least 80% identical to SEQ ID NO. 11.

15. A method according to claim 14, wherein the sequence of the introduced nucleic acid is identical to SEQ ID NO. 11.

16. A method according to claim 1, wherein the gene is ICE1 and the sequence of the introduced nucleic acid is at least 80% identical to SEQ ID NO. 3.

17. A method according to claim 16, wherein the sequence of the introduced nucleic acid is identical to SEQ ID NO. 3.

18. A vector for transforming a plant so as to provide the transformed plant with enhanced tolerance to an abiotic stress compared to an untransformed plant, the vector comprising at least one nucleic acid encoding a gene selected from the group consisting of OTS1, OTS2 and ICE1 under the control of a drought inducible promoter, wherein the nucleic acid has a sequence which is at least 80% identical to SEQ ID NO. 3, SEQ ID. NO 9 or SEQ ID NO. 11, respectively.

19. A vector according to claim 18, wherein the drought inducible is the Rab17 promoter (SEQ ID NO. 2).

20. A transformed plant or plant part produced according to the method of claim 1.


SEQUENCE LISTING (11 sequences)

SEQ ID NO: 1 - DNA, 2131 bp, Artificial Sequence
  Rab17 promoter and ICE1 gene coding sequence
gttgccgtactggtcgacatggcccgtggcgccgtggccgcgctgcccctgctgaccgtactccatgatgcttgcacggcttggtggtttgtggtttctcctaaacgagtcttcgtgcttgtgtgctcgatcgtgttctcgatgtggctcgccggtggtgaagcaggtgacggtcgatgccgccatttataggcagcagaagtgcagaacggcgaaaggattaggatctgggcgcgccacggcaggggtgtacgtgggcatgcatgagtggattgggcggccagtgagggaacagcacgtacacgtacgccggcgggacgtgtgcagtcgctggtgccgtgcgtcggtggcccggcccgccacgtctcggttcttctcgaaagttgggtacgcggattttaccggtgctcatccattattcaatgcaggcggtcggtcaggctgaatttaggcccagtacacccaagtgtaggcgcagcagaaaagtccagagcgaactgcataacattttaaaatacattaaaattcataaaatatattaaaagaagagaaaacattaaaaagtaaaatgataattcgataatgccaactattttaaaacaggatggtatagtatctcaggagtaccaattaggacatgtttgttaatgggtcttgacggaaacaatggtggaggggtttggttaaacggtggtggtggagaaagggaagagaacgaggaaggttcatggggaaggaatcaagaagatggttcttctcagtttaagcctatgcttgaaggtgattggtttagtagtaaccaaccacatccacaagatcttcagatgttacagaatcagccagatttcagatacttggtggttttccttttaaccctaatgataatcttcttcttcaacactctattgattcttcttcttcttgttctccttctcaagcttttagtcttgacccttctcagcaaaatcagttcttgtcaactaacaacaacaagggttgtcttctcaatgttccttcttctgcaaacccttttgataatgcttttgagtttggctctgaatctggttttcttaaccaaatccatgctcctatttcgatggggtttggttctttgacacaattggggaacagggatttgagttctgttcctgatttcttgtctgctcggtcacttcttgcgccggaaagcaacaacaacaacacaatgttgtgtggtggtttcacagctccgttggagttggaaggttttggtagtcctgctaatggtggttttgttgggaacagagcgaaagttctgaagcctttagaggtgttagcatcgtctggtgcacagcctactctgttccagaaacgtgcagctatgcgtcagagctctggaagcaaaatgggaaattcggagagttcgggaatgaggaggtttagtgatgatggagatatggatgagactgggattgaggtttctgggttgaactatgagtctgatgagataaatgagagcggtaaagcggctgagagtgttcagattggaggaggaggaaagggtaagaagaaaggtatgcctgctaagaatctgatggctgagaggagaaggaggaagaagcttaatgataggctttatatgcttagatcagttgtccccaagatcagcaaaatggatagagcatcaatacttggagatgcaattgattatctgaaggaacttctacaaaggatcaatgatcttcacaatgaacttgagtcaactcctcctggatctttgcctccaacttcatcaagcttccatccgttgacacctacaccgcaaactctttcttgtcgtgtcaaggaagagttgtgtccctcttctttaccaagtcctaaaggccagcaagctagagttgaggttagattaagggaaggaagagcagtgaacattcatatgttctgtggtcgtagaccgggtctgttgctcgctaccatgaaagctttggataatcttggattggatgttcagcaagctgtgatcagctgttttaatgggtttgccttggatgttttccgcgctgagcaatgccaagaaggacaagagatactgcctgatcaaatcaaagcagtgcttttcgatacagcagggtatgctggtatgatctga

SEQ ID NO: 2 - DNA, 647 bp, Maize
gttgccgtactggtcgacatggcccgtggcgccgtggccgcgctgcccctgctgaccgtactccatgatgcttgcacggcttggtggtttgtggtttctcctaaacgagtcttcgtgcttgtgtgctcgatcgtgttctcgatgtggctcgccggtggtgaagcaggtgacggtcgatgccgccatttataggcagcagaagtgcagaacggcgaaaggattaggatctgggcgcgccacggcaggggtgtacgtgggcatgcatgagtggattgggcggccagtgagggaacagcacgtacacgtacgccggcgggacgtgtgcagtcgctggtgccgtgcgtcggtggcccggcccgccacgtctcggttcttctcgaaagttgggtacgcggattttaccggtgctcatccattattcaatgcaggcggtcggtcaggctgaatttaggcccagtacacccaagtgtaggcgcagcagaaaagtccagagcgaactgcataacattttaaaatacattaaaattcataaaatatattaaaagaagagaaaacattaaaaagtaaaatgataattcgataatgccaactattttaaaacaggatggtatagtatctcaggagtaccaattaggacatgtttgtta

SEQ ID NO: 3 - DNA, 1484 bp, Arabidopsis thaliana
atgggtcttgacggaaacaatggtggaggggtttggttaaacggtggtggtggagaaagggaagagaacgaggaaggttcatggggaaggaatcaagaagatggttcttctcagtttaagcctatgcttgaaggtgattggtttagtagtaaccaaccacatccacaagatcttcagatgttacagaatcagccagatttcagatacttggtggttttccttttaaccctaatgataatcttcttcttcaacactctattgattcttcttcttcttgttctccttctcaagcttttagtcttgacccttctcagcaaaatcagttcttgtcaactaacaacaacaagggttgtcttctcaatgttccttcttctgcaaacccttttgataatgcttttgagtttggctctgaatctggttttcttaaccaaatccatgctcctatttcgatggggtttggttctttgacacaattggggaacagggatttgagttctgttcctgatttcttgtctgctcggtcacttcttgcgccggaaagcaacaacaacaacacaatgttgtgtggtggtttcacagctccgttggagttggaaggttttggtagtcctgctaatggtggttttgttgggaacagagcgaaagttctgaagcctttagaggtgttagcatcgtctggtgcacagcctactctgttccagaaacgtgcagctatgcgtcagagctctggaagcaaaatgggaaattcggagagttcgggaatgaggaggtttagtgatgatggagatatggatgagactgggattgaggtttctgggttgaactatgagtctgatgagataaatgagagcggtaaagcggctgagagtgttcagattggaggaggaggaaagggtaagaagaaaggtatgcctgctaagaatctgatggctgagaggagaaggaggaagaagcttaatgataggctttatatgcttagatcagttgtccccaagatcagcaaaatggatagagcatcaatacttggagatgcaattgattatctgaaggaacttctacaaaggatcaatgatcttcacaatgaacttgagtcaactcctcctggatctttgcctccaacttcatcaagcttccatccgttgacacctacaccgcaaactctttcttgtcgtgtcaaggaagagttgtgtccctcttctttaccaagtcctaaaggccagcaagctagagttgaggttagattaagggaaggaagagcagtgaacattcatatgttctgtggtcgtagaccgggtctgttgctcgctaccatgaaagctttggataatcttggattggatgttcagcaagctgtgatcagctgttttaatgggtttgccttggatgttttccgcgctgagcaatgccaagaaggacaagagatactgcctgatcaaatcaaagcagtgcttttcgatacagcagggtatgctggtatgatctga

SEQ ID NO: 4 - DNA, 2016 bp, Arabidopsis thaliana
atgacgaagaggaagaaggaagtaatagatgtcgattgctccgagaagaaagattttgtgattgattggtcttccgctatggataaggaagacgaagttcccgagctcgagattgttaataccaccaaacctactcctccgccaccgccaacgtttttctccgacgatcaaaccgattctccgaaactcctaaccgatcgtgacctcgacgagcagctagagcgtaaaaaagcgatcctgacattaggtccgggcttacccgacaagggtgagaaaattcgactcaaaatcgctgatctcgaagaggagaagcagcgtagagttttagaaggctcgaaaatggaggtggacagaagttctaaggtcgtgtcttcgactagctcaggttcagatgttttaccgcaaggaaatgcagtttcaaaagatacctctagagggaatgcagactcaaaagacacctctagacaagggaatgcagattcaaaagaagtctcacggtcaacattttctgcggttttcagtaaaccaaaaacggattctcagtcaaagaaagcctttggtaaagaactagaagatctgggatgtgaaaggaggaaacacaaggctggtagaaagcctgtaacaaggctgagcaacgggtggcggttgttgccagatgtagggaaagctgagcacagtgcaaagcagtttgattctggacttaaagaatcaaaagggaataagaaatccaaggaaccttatggaaagaaaaggcccatggaatcttcgacttattctctgattgatgatgatgatgatgatgatgatgatgatgacaacgacaccagtggccatgaaactcctagggagtggtcttgggaaaaatctccatcacaaagttcaaggcgccgtaagaaatcagaggacacagtgataaatgtggatgaagaagaagctcagccttcaacagtggcggagcaagcggctgaactgcctgaaggcttacaggaagatatatgctacccaacaagggatgatcctcactttgttcaagtttgtcttaaagatcttgaatgccttgcacctcgagaatatctgacatcgccggttatgaatttctacatgaggttcttgcagcagcagatatcatcatcgaatcaaatctctgctgattgtcacttctttaatacctatttctacaagaagctcagtgacgctgttacgtacaaggggaatgacaaggatgccttctttgtgaggttcaggcggtggtggaagggtattgatctatttcgtaaggcttatattttcataccaatacatgaagatctccactggagccttgtgatagtttgcatccctgataagaaagatgaatcggggttgactatacttcaccttgattctctaggacttcactcgagaaaatcaattgttgaaaatgtaaaaaggtttctaaaagacgaatggaattatttgaatcaagatgactattccttggatctgcctatctcagaaaaagtatggaaaaacctccctcgtaggatcagcgaagctgttgttcaggttccgcagcagaaaaacgattttgattgtggtccgtttgtgctcttcttcattaaacggttcattgaagaggcgcctcaaaggctgaaaaggaaagacctgggaatgttcgacaagaagtggtttagacccgatgaagcctctgctctgagaatcaaaatccgaaacacgctcatcgagctattccgtgtcagtgaccagacagagtaaaccagtacagattatgcagcttcaaatcaagacatttctcaccgctctaacagagttcaagtttttgttcttgtatagacttggcagaagtattatttgggccaaaagttaaattaaaaaatggctacagactccataggttaatgttgtaatccaaaaaaaaaaaatacctttgaacttttaattgtagtttcacaaaatttcattgttaaagaatctcagaagaagcataaaaaattggcatctaataagagttacgtt

SEQ ID NO: 5 - DNA, 2046 bp, Arabidopsis thaliana
atcaaaaaaaaagtttcaatttttgaaagctctgagaaatgaatctatcattctctctctctatctctatcttccttttcagatttcgcttcttcaattcatgaaatcctcgtgattctactttaatgcttctctttttttacttttccaagtctctgaatattcaaagtatatatcttttgttttcaaacttttgcagaattgtcttcaagcttccaaatttcagttaaaggtctcaactttgcagaattttcctctaaaggttcagactttggggtaaaggtgtcaactttggcgatgggtcttgacggaaacaatggtggaggggtttggttaaacggtggtggtggagaaagggaagagaacgaggaaggttcatggggaaggaatcaagaagatggttcttctcagtttaagcctatgcttgaaggtgattggtttagtagtaaccaaccacatccacaagatcttcagatgttacagaatcagccagatttcagatactttggtggttttccttttaaccctaatgataatcttcttcttcaacactctattgattcttcttcttcttgttctccttctcaagcttttagtcttgacccttctcagcaaaatcagttcttgtcaactaacaacaacaagggttgtcttctcaatgttccttcttctgcaaacccttttgataatgcttttgagtttggctctgaatctggttttcttaaccaaatccatgctcctatttcgatggggtttggttctttgacacaattggggaacagggatttgagttctgttcctgatttcttgtctgctcggtcacttcttgcgccggaaagcaacaacaacaacacaatgttgtgtggtggtttcacagctccgttggagttggaaggttttggtagtcctgctaatggtggttttgttgggaacagagcgaaagttctgaagcctttagaggtgttagcatcgtctggtgcacagcctactctgttccagaaacgtgcagctatgcgtcagagctctggaagcaaaatgggaaattcggagagttcgggaatgaggaggtttagtgatgatggagatatggatgagactgggattgaggtttctgggttgaactatgagtctgatgagataaatgagagcggtaaagcggctgagagtgttcagattggaggaggaggaaagggtaagaagaaaggtatgcctgctaagaatctgatggctgagaggagaaggaggaagaagcttaatgataggctttatatgcttagatcagttgtccccaagatcagcaaaatggatagagcatcaatacttggagatgcaattgattatctgaaggaacttctacaaaggatcaatgatcttcacaatgaacttgagtcaactcctcctggatctttgcctccaacttcatcaagcttccatccgttgacacctacaccgcaaactctttcttgtcgtgtcaaggaagagttgtgtccctcttctttaccaagtcctaaaggccagcaagctagagttgaggttagattaagggaaggaagagcagtgaacattcatatgttctgtggtcgtagaccgggtctgttgctcgctaccatgaaagctttggataatcttggattggatgttcagcaagctgtgatcagctgttttaatgggtttgccttggatgttttccgcgctgagcaatgccaagaaggacaagagatactgcctgatcaaatcaaagcagtgcttttcgatacagcagggtatgctggtatgatctgatctgatcctgacttcgagtccattaagcatctgttgaagcagagctagaagaactaagtccctttaaatctgcaattttcttctcaactttttttcttatgtcataacttcaatctaagcatgtaatgcaattgcaaatgagagttgtttttaaattaagcttttgagaacttgaggttgttgttgttggatacataacttcaaccttttattagcaatgttaacttccatttatgtttcatcttaaagctatgctcaagaatt

SEQ ID NO: 6 - DNA, 1943 bp, Arabidopsis thaliana
gaaagatttcagaccgaaacctaaataatattgagtaagtttattcgaagaagagatgaagagacaaagagcaatcgagttagatcgagtgaagaagacaatgttgaacatagattgggacgatgctttgggcgatgaggaagttcccgagctcgagatcatcgctactgacaaaattccgccgcgtgaacctactctttccggctatgaacccgccgtctctgtgcgatccctcagggataacgaactcgacgatcatctgaagcgtcagagatcacttcttactcgtctcggtgataagttggcagacaagggcgagaaaatccgaaacagaatcggagagctcgagtacgagaagcagcgaaggatgttccaacaacggaccaaaatggatgcagacaacggatgtcagattctcgagaaaccaaaaagctcagatgtgtttatgcgagcgagtacagcctcaaaagacacctctggacaagggacttcgggatcgaaagacgtgtctcggtcaacatttgctgctcattttagtgacaatctcaaaatgggacctcaaccagtgaaactagttaatgacaaactacaagatttgggacgtggaagctggataagcaaagctaatagagactcaataatagagaaaaataatgtgtggcggtcgctgccaagattaagcaagtgtaaggtcagtttaaagaacttttattctgagtctaaggatcctaaaggggatcgaagacccaatgaagcttatggtaagggaaagccaaatgaatcttccccttatttacttgtcgacgatgatgatggcgatgacgacaaagtcattggttatgaaactcccaggcattggagtttaaaagcatcgccactacagagttcaagctgcaggaagaaatcagatgataaagtgattaatttggatgaagatgaacctctgtctccaatggtagtagaggaagcatgtgaacttcctgaagggttaccagaagatatttactacccatcaagtgatcaaagtgatgggcgagaccttgttcaagtatctcttaaagatctgaaatgcctttcacctggggaatatcttacatcgccagttataaatttctacatcaggtacgtgcaacatcacgtgttttcagctgataagactgctgctaattgtcatttcttcaatacgtttttctacaagaagctcacagaagctgtttcatacaagggtaatgacagggatgcatattttgtgaagttcaggcggtggtggaagggttttgatctattctgtaaatcatatatatttataccaatacatgaagatcttcactggagcttagtcataatttgcatcccagacaaggaggacgaatcgggattgactataattcacttggattcattgggacttcacccaagaaatttgattttcaataatgtcaaaagatttctgagagaggaatggaactatctaaatcaagatgctccattggatttaccaatttcagcaaaagtatggagagaccttcccaatatgatcaacgaagctgaagtgcaggttccacaacagaagaacgatttcgactgtggtctgtttctgctcttcttcataagacgtttcatcgaagaggctcctcaaaggctgacattgcaggatttgaaaatgattcacaagaagtggtttaaaccggaagaagcttccgctttgaggatcaaaatctggaacatactcgttgatctattccgcaagggtaaccaaacagattaaagctgtatagaacagtgtcaagtttggtcactctaattaaggaatgaaatgaaagatacgtttttgttaacaatcattttgttcaagtcctgtaataaactaaggagcatgacataatcagaacaaatcaacaaggtaataataacgtatccgaataaaaagccaacattgctgc

SEQ ID NO: 7 - DNA, 21 bp, Artificial Sequence
  Primer
atacgctatttatttgcttgg

SEQ ID NO: 8 - DNA, 23 bp, Artificial Sequence
  Primer
ctggtttactctgtctggtcact

SEQ ID NO: 9 - DNA, 1760 bp, Arabidopsis thaliana
atgacgaagaggaagaaggaagtaatagatgtcgattgctccgagaagaaagattttgtgattgattggtcttccgctatggataaggaagacgaagttcccgagctcgagattgttaataccaccaaacctactcctccgccaccgccaacgtttttctccgacgatcaaaccgattctccgaaactcctaaccgatcgtgacctcgacgagcagctagagcgtaaaaaagcgatcctgacattaggtccgggcttacccgacaagggtgagaaaattcgactcaaaatcgctgatctcgaagaggagaagcagcgtagagttttagaaggctcgaaaatggaggtggacagaagttctaaggtcgtgtcttcgactagctcaggttcagatgttttaccgcaaggaaatgcagtttcaaaagatacctctagagggaatgcagactcaaaagacacctctagacaagggaatgcagattcaaaagaagtctcacggtcaacattttctgcggttttcagtaaaccaaaaacggattctcagtcaaagaaagcctttggtaaagaactagaagatctgggatgtgaaaggaggaaacacaaggctggtagaaagcctgtaacaaggctgagcaacgggtggcggttgttgccagatgtagggaaagctgagcacagtgcaaagcagtttgattctggacttaaagaatcaaaagggaataagaaatccaaggaaccttatggaaagaaaaggcccatggaatcttcgacttattctctgattgatgatgatgatgatgatgatgatgatgatgacaacgacaccagtggccatgaaactcctagggagtggtcttgggaaaaatctccatcacaaagttcaaggcgccgtaagaaatcagaggacacagtgataaatgtggatgaagaagaagctcagccttcaacagtggcggagcaagcggctgaactgcctgaaggcttacaggaagatatatgctacccaacaagggatgatcctcactttgttcaagtttgtcttaaagatcttgaatgccttgcacctcgagaatatctgacatcgccggttatgaatttctacatgaggttcttgcagcagcagatatcatcatcgaatcaaatctctgctgattgtcacttctttaatacctatttctacaagaagctcagtgacgctgttacgtacaaggggaatgacaaggatgccttctttgtgaggttcaggcggtggtggaagggtattgatctatttcgtaaggcttatattttcataccaatacatgaagatctccactggagccttgtgatagtttgcatccctgataagaaagatgaatcggggttgactatacttcaccttgattctctaggacttcactcgagaaaatcaattgttgaaaatgtaaaaaggtttctaaaagacgaatggaattatttgaatcaagatgactattccttggatctgcctatctcagaaaaagtatggaaaaacctccctcgtaggatcagcgaagctgttgttcaggttccgcagcagaaaaacgattttgattgtggtccgtttgtgctcttcttcattaaacggttcattgaagaggcgcctcaaaggctgaaaaggaaagacctgggaatgttcgacaagaagtggtttagacccgatgaagcctctgctctgagaatcaaaatccgaaacacgctcatcgagctattccgtgtcagtgaccagacagagtaaaccag

SEQ ID NO: 10 - DNA, 1485 bp, Arabidopsis thaliana
atgggtcttgacggaaacaatggtggaggggtttggttaaacggtggtggtggagaaagggaagagaacgaggaaggttcatggggaaggaatcaagaagatggttcttctcagtttaagcctatgcttgaaggtgattggtttagtagtaaccaaccacatccacaagatcttcagatgttacagaatcagccagatttcagatactttggtggttttccttttaaccctaatgataatcttcttcttcaacactctattgattcttcttcttcttgttctccttctcaagcttttagtcttgacccttctcagcaaaatcagttcttgtcaactaacaacaacaagggttgtcttctcaatgttccttcttctgcaaacccttttgataatgcttttgagtttggctctgaatctggttttcttaaccaaatccatgctcctatttcgatggggtttggttctttgacacaattggggaacagggatttgagttctgttcctgatttcttgtctgctcggtcacttcttgcgccggaaagcaacaacaacaacacaatgttgtgtggtggtttcacagctccgttggagttggaaggttttggtagtcctgctaatggtggttttgttgggaacagagcgaaagttctgaagcctttagaggtgttagcatcgtctggtgcacagcctactctgttccagaaacgtgcagctatgcgtcagagctctggaagcaaaatgggaaattcggagagttcgggaatgaggaggtttagtgatgatggagatatggatgagactgggattgaggtttctgggttgaactatgagtctgatgagataaatgagagcggtaaagcggctgagagtgttcagattggaggaggaggaaagggtaagaagaaaggtatgcctgctaagaatctgatggctgagaggagaaggaggaagaagcttaatgataggctttatatgcttagatcagttgtccccaagatcagcaaaatggatagagcatcaatacttggagatgcaattgattatctgaaggaacttctacaaaggatcaatgatcttcacaatgaacttgagtcaactcctcctggatctttgcctccaacttcatcaagcttccatccgttgacacctacaccgcaaactctttcttgtcgtgtcaaggaagagttgtgtccctcttctttaccaagtcctaaaggccagcaagctagagttgaggttagattaagggaaggaagagcagtgaacattcatatgttctgtggtcgtagaccgggtctgttgctcgctaccatgaaagctttggataatcttggattggatgttcagcaagctgtgatcagctgttttaatgggtttgccttggatgttttccgcgctgagcaatgccaagaaggacaagagatactgcctgatcaaatcaaagcagtgcttttcgatacagcagggtatgctggtatgatctga

SEQ ID NO: 11 - DNA, 1713 bp, Arabidopsis thaliana
atgaagagacaaagagcaatcgagttagatcgagtgaagaagacaatgttgaacatagattgggacgatgctttgggcgatgaggaagttcccgagctcgagatcatcgctactgacaaaattccgccgcgtgaacctactctttccggctatgaacccgccgtctctgtgcgatccctcagggataacgaactcgacgatcatctgaagcgtcagagatcacttcttactcgtctcggtgataagttggcagacaagggcgagaaaatccgaaacagaatcggagagctcgagtacgagaagcagcgaaggatgttccaacaacggaccaaaatggatgcagacaacggatgtcagattctcgagaaaccaaaaagctcagatgtgtttatgcgagcgagtacagcctcaaaagacacctctggacaagggacttcgggatcgaaagacgtgtctcggtcaacatttgctgctcattttagtgacaatctcaaaatgggacctcaaccagtgaaactagttaatgacaaactacaagatttgggacgtggaagctggataagcaaagctaatagagactcaataatagagaaaaataatgtgtggcggtcgctgccaagattaagcaagtgtaaggtcagtttaaagaacttttattctgagtctaaggatcctaaaggggatcgaagacccaatgaagcttatggtaagggaaagccaaatgaatcttccccttatttacttgtcgacgatgatgatggcgatgacgacaaagtcattggttatgaaactcccaggcattggagtttaaaagcatcgccactacagagttcaagctgcaggaagaaatcagatgataaagtgattaatttggatgaagatgaacctctgtctccaatggtagtagaggaagcatgtgaacttcctgaagggttaccagaagatatttactacccatcaagtgatcaaagtgatgggcgagaccttgttcaagtatctcttaaagatctgaaatgcctttcacctggggaatatcttacatcgccagttataaatttctacatcaggtacgtgcaacatcacgtgttttcagctgataagactgctgctaattgtcatttcttcaatacgtttttctacaagaagctcacagaagctgtttcatacaagggtaatgacagggatgcatattttgtgaagttcaggcggtggtggaagggttttgatctattctgtaaatcatatatatttataccaatacatgaagatcttcactggagcttagtcataatttgcatcccagacaaggaggacgaatcgggattgactataattcacttggattcattgggacttcacccaagaaatttgattttcaataatgtcaaaagatttctgagagaggaatggaactatctaaatcaagatgctccattggatttaccaatttcagcaaaagtatggagagaccttcccaatatgatcaacgaagctgaagtgcaggttccacaacagaagaacgatttcgactgtggtctgtttctgctcttcttcataagacgtttcatcgaagaggctcctcaaaggctgacattgcaggatttgaaaatgattcacaagaagtggtttaaaccggaagaagcttccgctttgaggatcaaaatctggaacatactcgttgatctattccgcaagggtaaccaaacagattaa